(19) United States
(12) Patent Application Publication
(10) Pub. No.: US 2008/0033503 A1
(43) Pub. Date: Feb. 7, 2008

(54) ELECTRODE CONFIGURATIONS FOR REDUCING INVASIVENESS AND/OR ENHANCING NEURAL STIMULATION EFFICACY, AND ASSOCIATED METHODS

(75) Inventors: Brad Fowler, Duvall, WA (US); Bradford Evan Gliner, Sammamish, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/845,005

(22) Filed: Aug. 24, 2007

Related U.S. Application Data
(62) Division of application No. 10/987,118, filed on Nov. 12, 2004.

(51) Int. Cl. A61N 1/04

(52) U.S. Cl. 607/45

(57) ABSTRACT

Electrode configurations for reducing invasiveness and/or enhancing neural stimulation efficacy, and associated methods, are disclosed. A method in accordance with one embodiment of the invention for treating a brain disorder includes identifying a target neural structure within a patient's skull and implanting an electrode device within the patient's skull so that an axis that is generally normal to the skull proximate to the electrode device and that passes through at least one electrical contact of the electrode device is offset from the target neural structure. The method further includes stimulating the target neural structure by applying an electrical signal to the at least one electrical contact. In particular embodiments, the electrode device can be positioned between, along, across, or adjacent to a fissure, recess, groove, and/or vascular structure of the patient's brain.

### TECHNICAL FIELD

The present disclosure describes particular types of electrode assemblies, electrode arrays, electrodes, electrical contacts, and/or signal transfer element configurations that may reduce surgical invasiveness and/or enhance neural stimulation efficacy.

### BACKGROUND

A wide variety of mental and physical processes are controlled or influenced by neural activity in particular regions of the brain. For example, the neural functions in some areas of the brain (e.g., the sensory or motor cortices) are organized according to physical or cognitive functions. There are also several other areas of the brain that appear to have distinct functions in most individuals. In the majority of people, for example, the areas of the occipital lobes relate to vision, the regions of the left interior frontal lobes relate to language, and the regions of the cerebral cortex appear to be consistently involved with conscious awareness, memory, and intellect.

Many problems or abnormalities with body functions can be caused by damage, disease and/or disorders in the brain. Effectively treating such abnormalities may be very difficult. For example, a stroke is a very common condition that damages the brain. Strokes are generally caused by emboli (e.g., obstruction of a vessel), hemorrhages (e.g., rupture of a vessel), or thrombi (e.g., clotting) in the vascular system of a specific region of the brain, which in turn generally cause a loss or impairment of a neural function (e.g., neural functions related to facial muscles, limbs, speech, etc.). Stroke patients are typically treated using various forms of physical therapy to rehabilitate the loss of function of a limb or another affected body part. Stroke patients may also be treated using physical therapy plus drug treatment. For most patients, however, such treatments are not sufficient, and little can be done to improve the function of an affected body part beyond the limited recovery that generally occurs naturally without intervention.

Neural activity in the brain can be influenced by electrical energy that is supplied by a waveform generator or other type of device. Certain patient perceptions and/or neural functions can thus be promoted or disrupted by applying an electrical current to the brain. As a result, researchers have attempted to treat particular neurological conditions using electrical stimulation signals to control or affect brain functions.

As an example, in deep brain stimulation, an electrode assembly coupled to a pulse system delivers electrical pulses to a deep brain region. For treatment of certain movement disorder symptoms, the deep brain region typically corresponds to the basal ganglia (e.g., the subthalamic nucleus). Unfortunately, implantation of an electrode assembly into a deep brain region involves a highly invasive surgical procedure.

Certain neural sites, locations, and/or populations may be more challenging to access than other neural regions. Notwithstanding, application of stimulation signals to such sites, locations, and/or populations may be desirable in view of increasing a likelihood of achieving a given stimulation result or therapeutic outcome. Unfortunately, conventional approaches for applying stimulation signals to such sites, locations, and/or populations may be undesirably invasive and/or result in undesirably limited neural stimulation efficacy.

### BRIEF DESCRIPTION OF THE DRAWINGS

FIG. 1A is a lateral illustration of the human brain.

FIG. 1B is a medial illustration of the human brain.

FIG. 1C is a top horizontal illustration of the human brain.

FIG. 1D is a coronal section through the right cerebral hemisphere illustrating certain topographic characteristics corresponding to the cerebral cortex.

FIG. 2 is a schematic diagram illustrating particular cortical vasculature of the brain.

FIG. 3 is a cross sectional illustration of the superior sagittal sinus and surrounding tissues located beneath the scalp and the skull.

FIG. 4A is a schematic illustration of a neural stimulation system implanted in a patient P according to an embodiment of the invention.

FIG. 4B is a longitudinal cross sectional illustration of an embodiment of a cross-structure implant configuration corresponding to FIG. 4A.

FIG. 5 is a schematic illustration showing an exemplary electric field distribution corresponding to the cross-structure implant configuration of FIG. 4B during unipolar electrical stimulation.

FIG. 6A is a schematic illustration showing an exemplary electric field distribution corresponding to the cross-structure implant configuration of FIG. 4B during bipolar electrical stimulation.

FIG. 6B is a schematic illustration showing particular cytoarchitectural characteristics of the cerebral cortex.

FIG. 7 is a lateral illustration identifying or generally identifying particular cortical areas or regions within the left hemisphere of the brain.

FIG. 8A is a schematic illustration of a neural stimulation system according to another embodiment of the invention.

FIG. 8B is a cross sectional schematic view of an intracranial electrode corresponding to the neural stimulation system of FIG. 8A.

FIG. 9 is a schematic illustration of a neural stimulation system having an articulated electrode assembly implanted in a patient to facilitate neural stimulation according to another embodiment of the invention.

FIG. 10 is a top isometric view of an electrode array according to an embodiment of the invention.

FIG. 11 is a flowchart illustrating an implantation and/or stimulation procedure according to an embodiment of the invention.

### DETAILED DESCRIPTION

The following disclosure describes various embodiments of systems and/or methods that may employ particular types of neural stimulators, electrode arrays, electrode assemblies, electrodes, and/or signal transfer element configurations to apply or deliver stimulation signals to and/or monitor neural activity associated with certain target neural populations, locations, sites, and/or structures. Such configurations may reduce surgical invasiveness and/or enhance the efficacy of a neural stimulation procedure.

Depending upon embodiment details and/or a type of neurologic dysfunction under consideration, a neural stimulation procedure may be directed toward facilitating and/or effectuating at least some degree of symptomatic relief and/or restoration or development of functional abilities in patients experiencing neurologic dysfunction arising from neurological damage, neurologic disease, neurodegenerative conditions, neuropsychiatric disorders, cognitive or learning disorders, and/or other conditions. Such neurologic dysfunction may correspond to Parkinson's Disease, essential tremor, Huntington's disease, stroke, traumatic brain injury, Cerebral Palsy, Multiple Sclerosis, a central pain syndrome, a memory disorder, dementia, Alzheimer's disease, an affective disorder, depression, bipolar disorder, anxiety, obsessive/compulsive disorder, Post Traumatic Stress Disorder, an eating disorder, schizophrenia, Tourette's Syndrome, Attention Deficit Disorder, an addiction, autism, epilepsy, a sleep disorder, an auditory or hearing disorder (e.g., tinnitus or auditory hallucinations), a speech disorder (e.g., stuttering), and/or one or more other disorders, states, or conditions.

In certain embodiments, a neural stimulation procedure may be initiated and/or performed in association and/or conjunction with an adjunctive and/or synergistic therapy procedure. An adjunctive and/or synergistic therapy may comprise, for example, one or more of a drug or chemical substance therapy; a neurotrophic and/or growth factor therapy; a cell implantation therapy; a behavioral therapy; and/or another type of therapy. Depending upon embodiment details, a behavioral therapy may comprise a physical therapy activity, a movement and/or balance exercise, a strength training activity, an activity of daily living (AbL), a vision exercise, a reading task, a speech task, a cognitive therapy, a memory or concentration task, a visualization or imagination exercise, a role playing activity, counseling, an auditory activity, an olfactory activity, a biofeedback activity, and/or another type of behavior, task, or activity that may be relevant to a patient's functional state, development, and/or recovery.

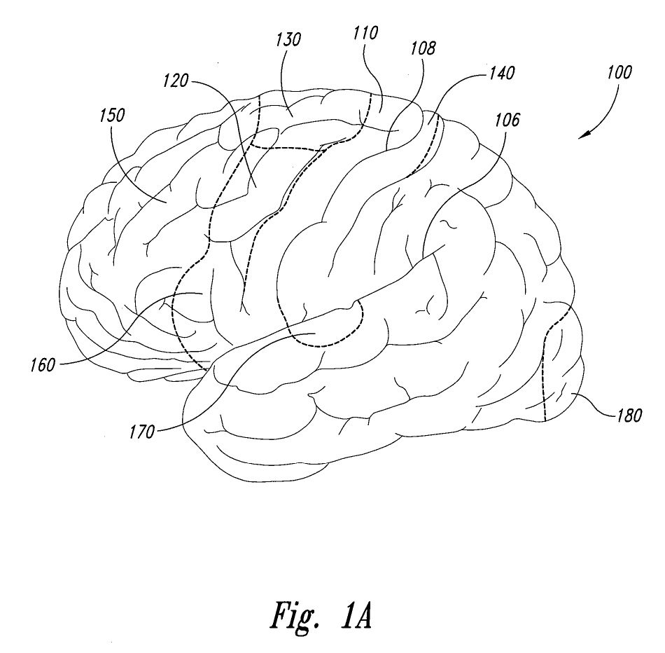

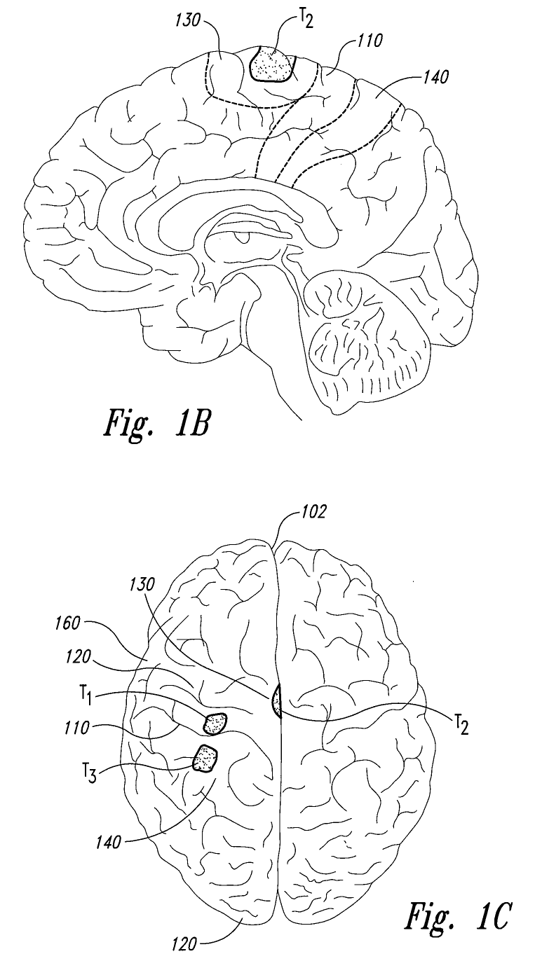

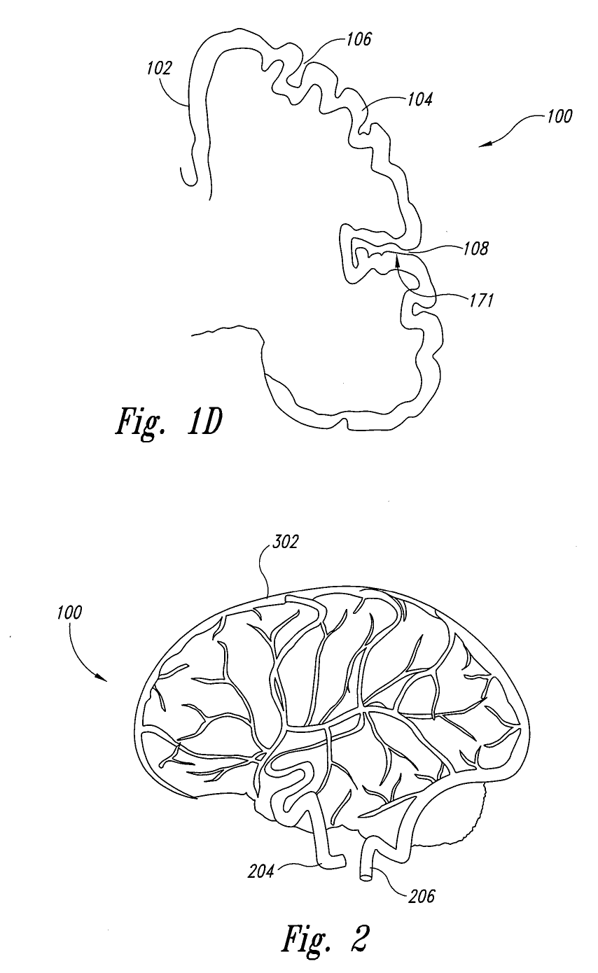

FIG. 1A is a lateral illustration, FIG. 1B is a medial illustration, and FIG. 1C is a top horizontal illustration of the human brain 100. Additionally, FIG. 1D is a coronal section through the right cerebral hemisphere illustrating certain topographic characteristics corresponding to the cerebral cortex or neocortex 104. In general, a target neural population may comprise a set, collection, group, and/or ensemble of neurons, neural structures, neural projections, and/or neural regions to which the application of stimulation signals may be desirable, for example, to influence, affect, and/or treat one or more types of neurologic dysfunction. Depending upon a type of neurologic dysfunction under consideration and/or embodiment details, one or more target neural populations may reside upon, within, and/or beneath one or more areas or regions of the neocortex 104. Such cortical areas may comprise and/or correspond to, for example, one or more portions of the motor cortex 110, the premotor cortex 120, the supplementary motor cortex (SMA) 130, the somatosensory cortex 140, the prefrontal cortex 150, Broca's area 160, the auditory cortex 170 (primary and/or secondary), the visual cortex 180, and/or one or more other cortical areas (e.g., Heschl's gyri 171, shown in FIG. 1D).

Any given target neural population may be involved in influencing and/or controlling one or more types of cognitive and/or physical functions or processes. Stimulating a target neural population may directly affect the functioning of that population or another population or structure that communicates with the target neural population. FIGS. 1A, 1B, and 1C illustrate certain representative target neural populations, namely, a target neural population T1 (FIG. 1C) corresponding to a portion of the motor cortex 110; a target neural population T2 (FIGS. 1B and 1C) corresponding to a portion of the SMA 130; and a target neural population T3 (FIG. 1C) corresponding to a portion of the somatosensory cortex 140.

In general, a stimulation site may be defined as an anatomical region or location at or near which stimulation signals may be applied to stimulate, affect, or influence at least a portion of one or more target neural populations. In the context of several embodiments described herein, a set of stimulation sites may correspond to one or more epidural and/or subdural cortical locations in one or both cerebral hemispheres.

A target neural population and/or a stimulation site may be identified and/or located in a variety of manners, for example, through one or more procedures involving neural imaging, electrophysiological signal measurement, and/or anatomical landmark identification. Exemplary manners of identifying a target neural population and/or a stimulation site are given in U.S. application Ser. No. 09/802,808, entitled “Methods and Apparatus for Effectuating a Lasting Change in a Neural-Function of a Patient”, filed on Mar. 8, 2001; and U.S. application Ser. No. 10/317,002, entitled “Systems and Methods for Enhancing or Optimizing Neural Stimulation Therapy for Treating Symptoms of Parkinson's Disease and/or Other Movement Disorders,”, filed on Dec. 10, 2002, each of which is incorporated herein by reference in its entirety.

Particular neuroanatomical structures may at least partially obstruct, obscure, conceal, overlay, encompass, and/or include one or more target neural populations in a manner that may complicate direct physical and/or electrical access to one or more portions of such neural populations. For example, as shown in FIGS. 1B and 1C, a portion of a target neural population T2 may reside upon or within a region of the SMA 130 that itself resides proximate to and/or along the crest of the interhemispheric fissure 102.

Several types of neuroanatomical structures may influence an extent to which the implantation of neural stimulation and/or monitoring devices at, proximate, or relative to a stimulation site may be considered invasive. Moreover, the presence of such neuroanatomical structures beneath or proximate to a stimulation site may affect neural stimulation efficacy. Neuroanatomical structures of interest may include cerebral topographical structures or features; cerebral vasculature; and/or other structures. Cerebral topographical features may be quite convoluted, and may include folds, grooves, openings, fissures, sulci, ridges, and/or gyri. Some of the major sulci, such as the lateral sulcus (or the Sylvian fissure) 106 and the central sulcus (or Rolandic fissure) 108 comprise large indentations on cortical surfaces.

In general, based upon size, diameter, and/or relative blood volume carrying capacity, individual vascular structures may be categorized as major vessels; sinuses; vascular trunks; vascular branches; fine vessels; and microvasculature. Certain embodiments of the invention involve the implantation, positioning, and/or placement of stimulation devices relative to particular types of vascular structures, such as major vessels, sinuses, vascular trunks, and/or vascular branches.

FIG. 2 is a schematic diagram illustrating particular major vessels, sinuses, vascular trunks, and/or vascular branches that may reside above, upon, adjacent to, and/or within the neocortex 104 (FIG. 1D). Multiple veins and arteries carry necessary substances for proper brain function. The arteries deliver oxygenated blood, glucose and other nutrients to the brain 100 while the veins remove de-oxygenated blood, carbon dioxide, and other metabolic products from the brain 100. The brain 100 receives as much as one-fifth of the blood pumped by the heart and consumes approximately twenty percent of the oxygen utilized by the body. Various types of vasculature are involved in exchanging blood with the brain 100. The blood is circulated completely through the brain 100 by way of a major input artery, the internal carotid artery 204, to a major output vessel, the internal jugular vein 206, all within about seven seconds. Both the right and left hemispheres are supplied by the internal carotid artery 204, which penetrates the dura and supplies the anterior, middle, and posterior cerebral arteries.

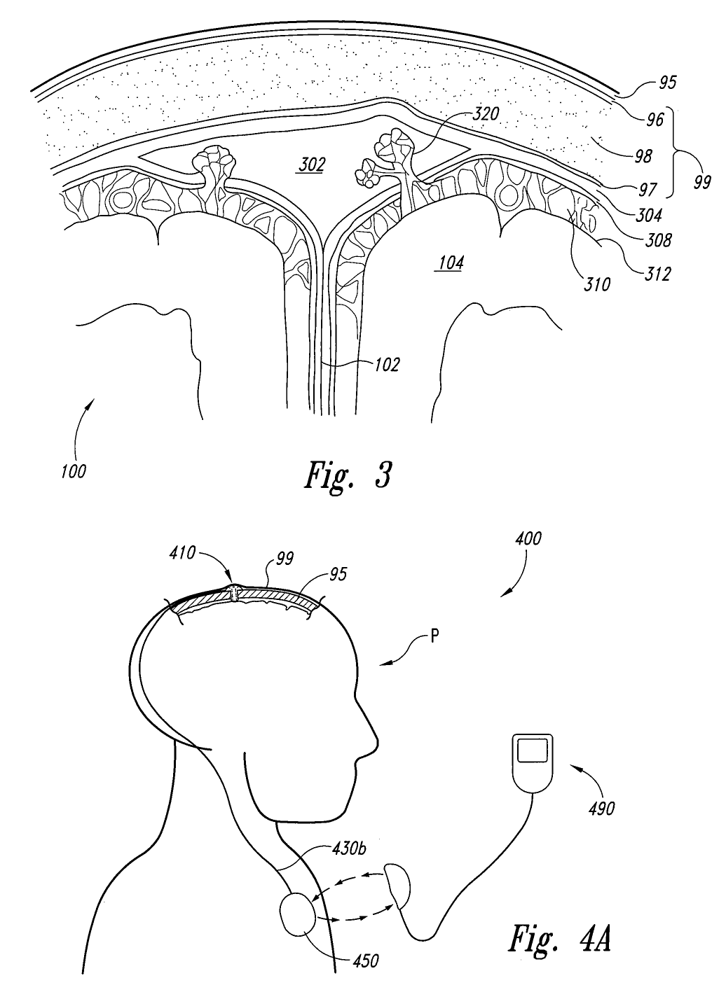

FIG. 3 is a cross sectional illustration of the superior sagittal sinus (SSS) 302 (which is also shown in side view in FIG. 2) and surrounding tissues located beneath the scalp 95 and the skull 99. The skull 99 includes the cancellous 98, located between the outer table 96 and the inner table 97. The SSS 302 comprises a long venous drainage channel, essentially spanning the length of the brain 100 along an anterior to posterior direction. Referring also now to FIG. 1C, the SSS 302 resides just above and/or partially along and/or within the crest of the interhemispheric fissure 102. The SSS 302 is embedded within the dura mater 304, which resides above the arachnoid mater 308, which resides above the subarachnoid cavity 310, which resides above the pia mater 312, which resides upon the surface of the cerebral cortex 104. Particular structures reside within the SSS 302, including the arachnoid granulations 320, which reabsorb cerebrospinal fluid.

Various embodiments of the invention are directed toward implanting, configuring, positioning, and/or orienting one or more neural stimulation devices such as electrode assemblies, electrode arrays, and/or signal transfer structures in a manner that may 1) enhance a likelihood of effectively applying stimulation signals to less readily accessible neural populations; and/or 2) reduce or minimize surgical invasiveness. Such electrode assemblies, electrode arrays, and/or signal transfer structures may include transcranial screw and/or peg electrode assemblies; articulated electrode arrays or assemblies; grid electrode structures; and/or other types of signal transfer structures, as described in detail hereafter.

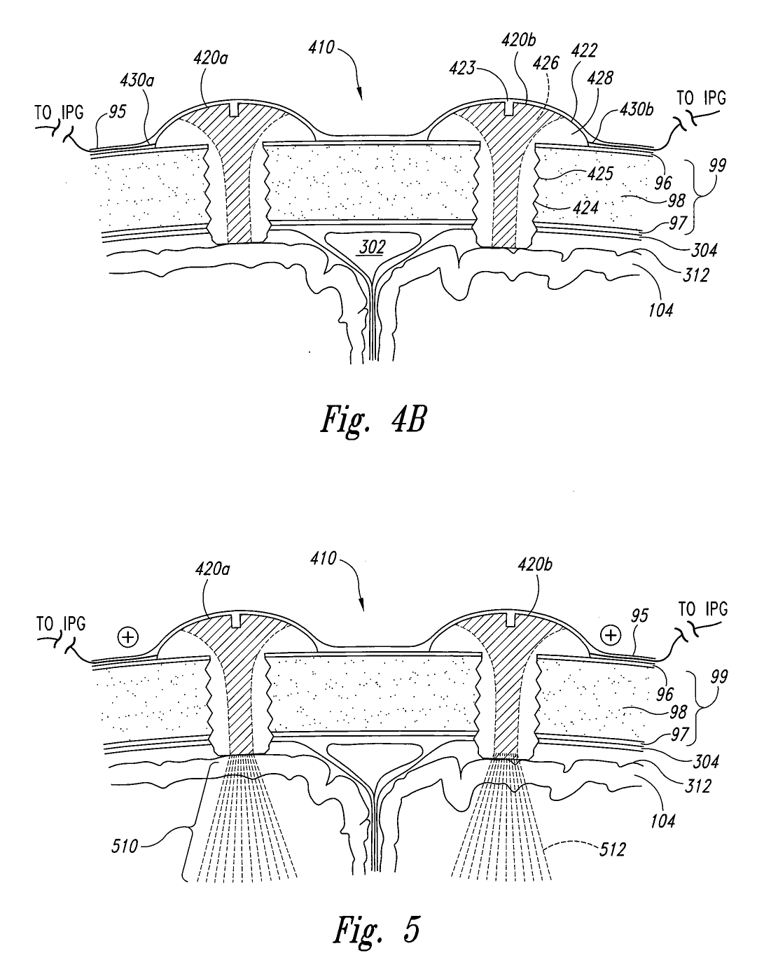

FIG. 4A is a schematic illustration of a neural stimulation system 400 implanted in a patient P according to an embodiment of the invention. FIG. 4B is a longitudinal cross sectional illustration of an embodiment of a cross-structure implant configuration 410 corresponding tQ FIG. 4A. Depending upon embodiment details, a cross-structure implant configuration 410 may comprise a set of neural stimulation devices implanted or positioned across, generally across, between, along, and/or relative to one or more neuroanatomical structures.

In one embodiment, the cross-structure implant configuration 410 comprises a set of transcranial screw electrode assemblies 420a, 420b implanted proximate to the SSS 302, where at least a first electrode assembly 420a corresponds to the left cerebral hemisphere and at least a second electrode assembly 420b corresponds to the right cerebral hemisphere. In another embodiment, each electrode assembly 420a, 420b or multiple electrode assemblies 420a, 420b may correspond to or reside within a single cerebral hemisphere.

Any given transcranial screw electrode assembly 420a, 420b may comprise a housing, body, and/or support structure that carries at least one electrical contact and/or signal transfer element that may serve as an electrical interface to neural tissue. In one embodiment, a transcranial screw electrode assembly 420a, 420b comprises a head 422 and a shaft 424 forming a body of the electrode assembly 420a, 420b. The electrode assembly 420a, 420b may include a conductive core 426 that facilitates transfer or conduction of electrical energy to and/or from a stimulation site. The conductive core 426 may be integrally formed using an electrically conductive biocompatible material, e.g., titanium, platinum, and/or another material. The conductive core 426 may be carried by an electrically insulating material 428, which may form one or more portions of the head 422 and/or shaft 424.

In some embodiments, the shaft 424 may include threads 425 for tapping the electrode assembly 420a, 420b into the skull 95 to a desired depth. In certain embodiments, the head 422 may include one or more slots 423, notches, grooves, recesses, bores, and/or other structures to facilitate such tapping. Various embodiments of neural stimulation systems and/or transcranial screw and/or peg electrode assemblies that may be suited to particular embodiments of the present invention are described in U.S. application Ser. No. 10/891,834, entitled “Methods and Systems for Intracranial Neurostimulation and/or Sensing,” filed on Jul. 15, 2004, which is incorporated herein in its entirety by reference.

Each electrode assembly 420a, 420b may be coupled by a lead wire or link 430a, 430b to a power source such as a pulse generator 450. The pulse generator 450 may be implanted in the patient P, for example, in a subclavicular location. In various embodiments, the pulse generator 450 may comprise an energy storage device, a programmable computer medium, signal generation circuitry, control circuitry, and/or other elements that facilitate the generation and output of stimulation signals, waveforms, or pulses to particular electrode assemblies 420a, 420b and/or signal transfer elements at one or more times. In certain embodiments, the pulse generator 450 may include additional circuitry for receiving, monitoring, and/or analyzing signals received from one or more implanted devices. An external programming unit 490 may communicate program instructions, stimulation signal parameters, patient-related data, and/or other information to the pulse generator 450, in a manner understood by those skilled in the art.

In one embodiment, each electrode assembly 420a, 420b may be implanted and/or approximately positioned a minimum distance away from a border, approximate border, and/or reference location corresponding to the SSS 302, other cerebral vasculature, and/or one or more other neuroanatomical structures. A minimum or approximate minimum implantation distance may reduce a likelihood of 1) affecting a neuroanatomical structure under consideration during or after a surgical procedure; and/or 2) routing, diverting, or shunting an undesirable amount of electrical current (e.g., an amount of current that may have a significant likelihood of reducing neural stimulation efficacy) through portions of cerebral vasculature during a neural stimulation procedure. Depending upon embodiment details and/or patient condition, a minimum lateral implantation distance relative to a border of the SSS 302, other cerebral vasculature, and/or one or more other neuroanatomical structures may be between about 0.5 and 2.0 mm, and in a particular embodiment, about 1.0 mm.

In some embodiments, each electrode assembly 420a, 420b may be implanted epidurally. In other embodiments, one or more electrode assemblies 420a, 420b may be implanted subdurally. In certain situations, a subdural electrode assembly 420a, 420b may facilitate transfer of electrical signals in a different or slightly different manner than an epidural electrode assembly 420a, 420b. While a given subdural implantation may be more invasive than a corresponding epidural implantation, a subdural implantation may be generally, relatively, or reasonably noninvasive (particularly with respect to, for example, implantation of an electrode assembly into a deep brain region). In general, whether an implant configuration 410 comprises epidural and/or subdural electrode assemblies 420a, 420b may depend upon embodiment details, intended stimulation signal path characteristics, the nature and/or extent of a patient's neurologic dysfunction, patient condition, and/or one or more other factors.

Stimulation site location, stimulation device characteristics, and/or simulation signal characteristics may determine an extent to which stimulation signals may reach, affect, and/or influence portions of a target neural population. In certain situations, neural stimulation efficacy may be affected through the application of stimulation signals having particular polarity characteristics. In various embodiments, electrode assemblies 420a, 420b may be configured to apply unipolar and/or bipolar stimulation signals to a stimulation site at one or more times.

During unipolar stimulation, a set of electrode assemblies 420a, 420b positioned relative to a stimulation site are biased such that each electrically active electrode assembly 420a, 420b has an identical polarity at any given time. Additionally, one or more conductive elements positioned remote from the stimulation site are biased at a ground, common, or opposite polarity to provide electrical path continuity. A remote conductive element may comprise, for example, an implanted electrode array, an implanted electrode assembly 420a, 420b, one or more portions of an implanted pulse generator's housing, and/or a surface or skin mounted electrode.

In a unipolar configuration, each electrode assembly 420a, 420b at a stimulation site may 1) serve as an anode, while the remote conductive element serves as a cathode; or 2) serve as a cathode, while the remote conductive element serves as an anode at any given time. In general, a stimulation signal may comprise a pulse, pulse series, and/or pulse train having multiple phases, where the polarities and/or other characteristics of the phases may vary. For example, a stimulation signal may comprise a biphasic pulse train, in which each pulse within the pulse train has a positive first phase and a negative second phase. In various embodiments, the terms “anode” and “cathode” may be defined relative to the polarity of a first or initial pulse phase. In one embodiment, an anodal unipolar configuration exists when each electrode assembly 420a, 420b at a stimulation site is configured to apply a positive (+) first pulse phase, while a remote conductive element is configured to complete a circuit path at a lower polarity (−) relative to each anode. Similarly, in one embodiment, a cathodal unipolar configuration exists when each electrode assembly 420a, 420b at a stimulation site is configured to apply a negative (−) first pulse phase, while a remote conductive element is configured to complete a circuit path at a higher polarity (+) relative to each cathode.

FIG. 5 is a schematic illustration showing an exemplary electric field distribution 510 corresponding to the cross-structure implant configuration 410 of FIG. 4B during unipolar electrical stimulation. Relative to FIGS. 4A and 4B, like reference numbers indicate like or generally like elements. In the embodiment shown and/or at a particular time, the first and second electrode assemblies 420a, 420b are each configured as an anode (+), while a portion of the pulse generator's housing and/or another remote conductive element coupled to the pulse generator 450 may be configured as a cathode (−). In another embodiment and/or at another time, the relative polarities of the anode (+) and the cathode (−) may be opposite.

The representative electric field distribution 510 may be illustrated by a plurality of electric field lines 512 extending from each anodal (+) electrode assembly 420a, 420b and extending along a path that includes, for example, the cathodal (−) pulse generator housing. One or more electric field lines 512 may correspond, generally correspond, or approximately correspond to an electric current path from the electrode assemblies 420a, 420b to the pulse generator's housing. In certain situations, unipolar stimulation may facilitate enhanced efficacy stimulation of deeper cortical and possibly subcortical tissues that may be reached or influenced by such a current path. Unipolar stimulation may alternatively or additionally facilitate enhanced development and/or recovery of functional abilities in patients experiencing particular types of neurologic dysfunction, in a manner identical, essentially identical, or analogous to that described in U.S. application Ser. No. 10/910,775, entitled “Apparatus and Methods for Applying Neural Stimulation to a Patient”, filed on Aug. 2, 2004, incorporated herein in its entirety by reference.

In addition or as an alternative to unipolar stimulation, particular embodiments of the invention may apply bipolar stimulation signals at one or more times. During bipolar stimulation, two or more electrode assemblies 420a, 420b positioned relative to a stimulation site are biased such that at least one electrode assembly 420a, 420b acts and an anode (+) and at least one electrode assembly 420a, 420b acts as a cathode (−) at any given time.

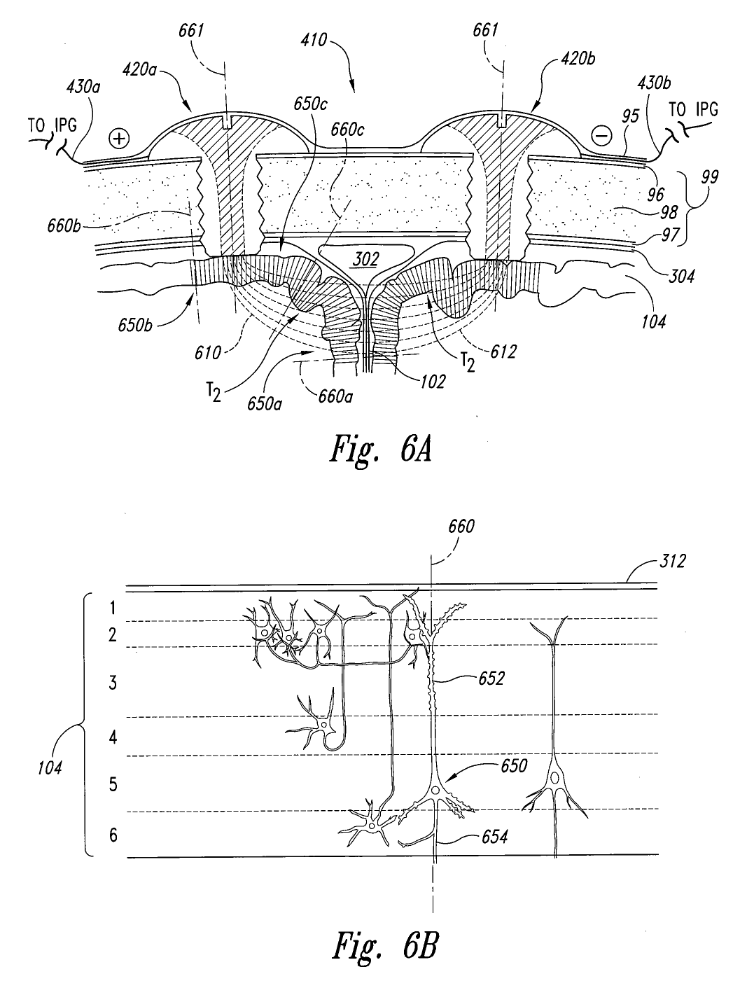

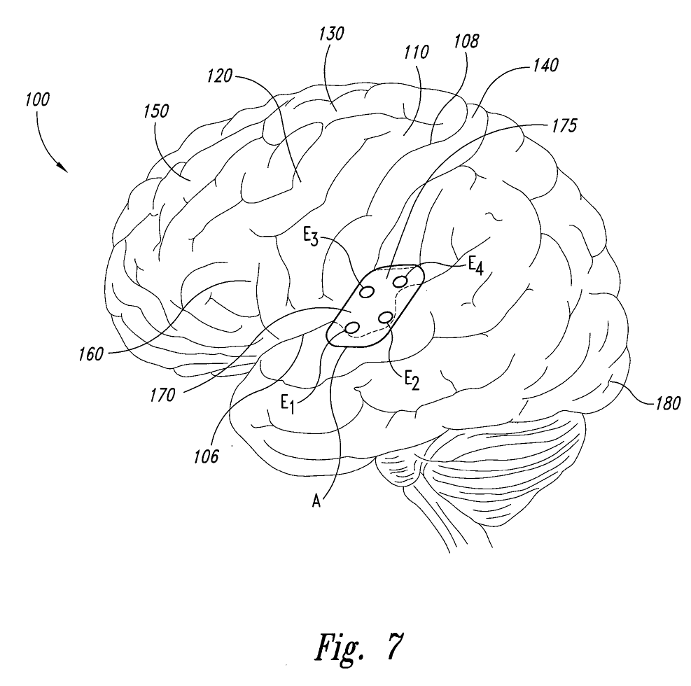

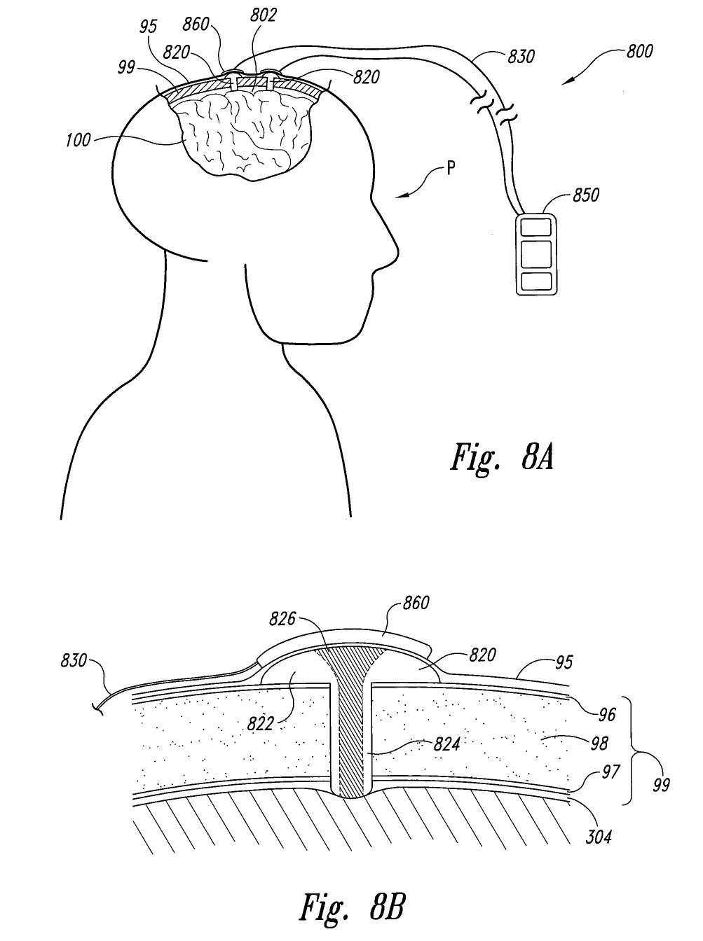

FIG. 6A is a schematic illustration showing a representative electric field distribution 610 corresponding to the cross-structure implant configuration 410 of FIG. 4B during bipolar electrical stimulation. Relative to FIG. 4B, like reference numbers indicate like or generally like elements. In the embodiment shown, the first electrode assembly 420a may be configured as an anode (+), while the second electrode assembly 420b may be configured as a cathode (−) at one or more times. In an alternate embodiment, the first electrode assembly 420a may be configured as a cathode (−), while the second electrode assembly 420b may be configured as an anode (+) at one or more times.

The representative electric field distribution 610 in FIG. 6A is illustrated by a plurality of electric field lines 612 that extend from the anode (+) to the cathode (−), and which may correspond, generally correspond, or approximately correspond to an electric current path between the first and second electrode assemblies 420a, 420b. As indicated in FIG. 6A, a bipolar configuration may facilitate stimulation of neural tissues that reside directly or generally beneath the first and second electrode assemblies 420a, 420b. Moreover, a bipolar configuration may facilitate stimulation of neural tissues that reside between the first and second electrode assemblies 420a, 420b in situations in which direct access to such neural tissues may be complicated by one or more neuroanatomical structures such as the SSS 302, the interhemispheric fissure 102, and/or other tissues or structures.

In various embodiments of the invention, the application of unipolar and/or bipolar stimulation signals may increase a likelihood of effectively stimulating particular types of neurons and/or neural structures that may be characterized by one or more types of spatial alignments and/or orientations relative to a set of externally consistent or invariant brain, head, and/or patient reference axes or directions. In general, relative to such reference axes or directions, an alignment or orientation of one or more types of cortical neurons located proximate to and/or within a fissure, recess, or groove may differ from that of corresponding types of cortical neurons located away from the fissure, recess or groove or upon a gyrus. Similarly, an alignment or orientation corresponding to one or more types of cortical neurons may change or vary with a distance defined relative to a vascular or other type of neuroanatomical structure, as further described below.

Cortical topography may vary depending upon proximity to particular neuroanatomical structures. As indicated in FIG. 6A, the cortex 104 curves proximate to the SSS 302, and follows a trajectory defined by the interhemispheric fissure 102. As a result, an alignment or orientation corresponding to particular types of intracortical structures within target neural population T2 may change or vary based upon proximity to the SSS 302 and/or the interhemispheric fissure 102. More specifically, with respect to a consistent and/or invariant brain, head, and/or patient reference coordinate system, particular types of cortical neurons within T2 that reside beneath the SSS 302 and/or within or along the interhemispheric fissure 102 may exhibit a different alignment or orientation than corresponding types of cortical neurons within portions of T2 that reside directly beneath or generally beneath the first and second electrode assemblies 420a, 420b, as further described hereafter.

FIG. 6B is a schematic illustration showing particular cytoarchitectural characteristics of the neocortex 104. The neocortex 104 ranges between approximately 1 and 4 mm in thickness, and generally exhibits a layer structure transverse to its thickness (i.e., a laminar structure). Typically, the layer structure is defined to include layers 1-6, where layer 1 originates at the cortical surface, and layer 6 terminates at a cortical-subcortical boundary. Pyramidal cells 650 within the neocortex 104 provide the principal neural output pathways that project to subcortical structures. A pyramidal cell body receives input and transmits output along a dendritic pathway 652 and an axonal pathway 654, respectively, that may define a signal transmission axis 660 that is generally perpendicular to the cortical layer structure and/or the pia mater 312.

Referring again to FIG. 6A, first pyramidal cells 650a within various portions of the neocortex 104 along the interhemispheric fissure 102 may exhibit or generally exhibit a medial-lateral alignment of first signal transmission axes 660a. In other words, the first pyramidal cells 650a can have signal transmission axes 660a that are generally perpendicular to the interhemispheric fissure 102. Second pyramidal cells 650b directly or approximately beneath the first and/or second electrode assemblies 420a, 420b may exhibit or generally exhibit a superior—inferior alignment of second signal transmission axes 660b, or a dendritic—axonal structural alignment that may be generally perpendicular to the skull 99. Finally, third pyramidal cells 650c within portions of the neocortex 104 along or proximate to the crest of the interhemispheric fissure 102 exhibit a structural alignment or orientation that is generally between the two aforementioned alignments. In particular, these cells can have third signal transmission axes 660c having angular orientations between the first signal transmission axes 660a and the second signal transmission axes 660b.

In one aspect of an embodiment shown in FIG. 6A, at least one of the electrodes 420a, 420 provides stimulation to target neural structures (e.g., first pyramidal cells 650a) that are offset from an axis 661 that is generally normal to the skull 99 and passes through the electrode. For example, as shown in FIG. 6A, the electrodes 420a, 420b can be deliberately offset from the first pyramidal cells 650a located between them to generate an electrical field that is aligned with the first transmission axes 660a of those cells. In the embodiment shown in FIG. 6A, the electrical field is also aligned with the second transmission axes 660b of second pyramidal cells 650b located directly beneath the electrodes 420a, 420b. In other embodiments, this need not be the case. In another aspect of an embodiment shown in FIG. 6A, the first pyramidal cells 650a located between the electrodes 420a, 420b can be located interior to the SSS 302. By offsetting the electrodes 420a, 420b from both the SSS 302 and the first pyramidal cells 650a, electrical signals can propagate to the first pyramidal cells 650a without interference from fluid in the SSS 302. In other embodiments, this approach can be used to direct electrical signals around other potentially interfering structures within the patient's skull 99. For example, this approach can be used to direct unipolar and/or bipolar stimulation signals to target areas located within a fissure or crevice via one or more electrodes positioned outside the fissure or crevice.

Certain neural stimulation procedures may be directed toward affecting particular pyramidal cell populations at one or more times, possibly in a preferential manner relative to other pyramidal cell populations, other types of neurons, and/or other neural structures. During a neural stimulation procedure, the application of unipolar and/or bipolar stimulation signals using a cross-structure implant configuration 410 may enhance an extent to which stimulation signals reach, influence, and/or affect pyramidal cells 650 and/or other neural structures that reside proximate to, at least partially within, beneath, and/or between one or more neuroanatomical structures that the cross-structure implant configuration 410 spans.

FIG. 7 is a lateral illustration identifying or generally identifying particular cortical areas or regions within the left hemisphere of the brain 100. Relative to FIGS. 1A-1D, like reference numbers may indicate or correspond to like cortical areas. While FIG. 7 depicts the brain's left hemisphere, various portions of the description that follows may alternatively or additionally apply to the right hemisphere of the brain 100 in an identical, essentially identical, and/or analogous manner.

In certain embodiments, it may be desirable to apply electrical stimulation signals at, within, proximate to, around, above, to, and/or through portions of at least one target area A (as indicated by shading) that may include 1) cortical surfaces and/or regions that are proximate to particular types of neuroanatomical structures (e.g., cerebral vasculature and/or topographical features such as gyri, folds, and/or fissures); and/or 2) portions of and/or projections into one or more cortical surfaces and/or structures that are less readily accessible and/or at least partially recessed, obstructed, or hidden as a result of such neuroanatomical structures. Depending upon embodiment details, a type of neurologic dysfunction under consideration, patient condition, and/or patient treatment history (which may relate to neural stimulation and/or other types of treatment), portions of one or more target areas A may reside in the same or different hemispheres.

In a representative embodiment, the target area A may comprise one or more target neural populations that are proximate to and/or at least partially within the lateral (Sylvian) fissure 106. For example, the target area A may comprise a cortical region corresponding to portions of the auditory cortex 170 and/or one or more neural populations that may have projections into, proximate to, and/or associated with the auditory cortex 170. In some embodiments, the target area A may additionally or alternatively comprise a cortical region corresponding to portions of the somatosensory cortex, for example, the secondary somatosensory cortex 175. The application or delivery of electrical stimulation signals to, within, and/or near portions of the auditory cortex 170, possibly in association with the simultaneous and/or sequential or alternating application or delivery of stimulation signals to, within, and/or near portions of the secondary somatosensory cortex 175, may facilitate the treatment of auditory neurologic dysfunction such as tinnitus and/or auditory hallucinations. Such stimulation may occur in a predetermined, aperiodic, and/or quasi-random manner. Certain embodiments may involve the simultaneous or alternating stimulation of homologous and/or nonhomologous sites in different brain hemispheres (e.g., the stimulation of one or more regions corresponding to the auditory cortex 170 in one hemisphere, in association with the stimulation of one or more regions corresponding to the secondary somatosensory cortex 175 in the other or both hemispheres). Depending upon embodiment details, stimulation of sites in different hemispheres may involve single or multiple pulse generating devices or systems. Other embodiments may be directed toward independent, simultaneous, or alternating stimulation of other and/or additional target areas.

A set of stimulation devices and/or signal transfer elements, for example, one or more devices shown in FIG. 7 as E1-E4, may be selectively placed and/or implanted within, about, above, proximate to, and/or relative to portions of a target area A. Particular stimulation devices E1-E4 may be located, oriented, and/or configured relative to particular neuroanatomical structures and/or each other in such a manner as to enhance a likelihood that the application of stimulation signals affects portions of the target area A and/or neural projections associated therewith in an intended manner. During a neural stimulation procedure, electrical energy may be applied, varied, and/or manipulated in particular manners to facilitate or enhance a likelihood of penetration and/or transfer of electrical signals into targeted tissue, possibly in a preferential or orientation dependent manner.

One or more of stimulation devices E1-E4 may be configured to apply bipolar stimulation signals and/or unipolar stimulation signals at one or more times. In one embodiment, for a target area A corresponding to portions of the auditory cortex 170 and possibly portions of the secondary somatosensory cortex 175, the application of bipolar and/or unipolar stimulation signals to particular stimulation devices E1-E4 at one or more times may enhance a likelihood of affecting neural populations that map to particular auditory processing functions (e.g., auditory signal perception, tone or timbre discrimination, spatial localization, noise filtering, and/or other functions). For example, the application of unipolar stimulation signals at one or more times may enhance a likelihood of affecting neural regions that tonotopically map to particular auditory frequencies and/or frequency ranges (e.g., in certain patients, unipolar stimulation may enhance the efficacy of neural stimulation directed toward treating tinnitus symptoms, possibly including symptoms associated with higher auditory frequencies).

In addition or as an alternative to the foregoing, one or more other stimulation parameters (e.g., a pulse repetition frequency, a first phase pulse width, a peak current or voltage amplitude, a burst or pulse packet frequency, a waveform modulation function, a duty cycle and/or a spatiotemporal stimulation signal delivery or stimulation device activation pattern, and/or another parameter) may be selected and/or varied at one or more times to affect neural stimulation efficiency and/or efficacy. In certain situations, a known, anticipated, or estimated range of stimulation parameters and/or stimulation parameter characteristics may influence the relative positions of one or more stimulation devices E1-E4. In general, one or more stimulation parameters such as those indicated herein may be varied in relation to one or more time domains (e.g., an hours-based, a seconds-based, and/or a subseconds-based time domain) in a predetermined, aperiodic, and/or quasi-random manner, possibly depending upon embodiment details, a type of neurologic dysfunction under consideration, patient condition, a length of time that neural stimulation has previously or recently been applied, previous stimulation parameter values, and/or other factors. Such parameter variation may enhance and/or maintain neural stimulation efficacy, and/or increase a time interval over which neural stimulation may provide a high, significant, or acceptable level of symptomatic relief.

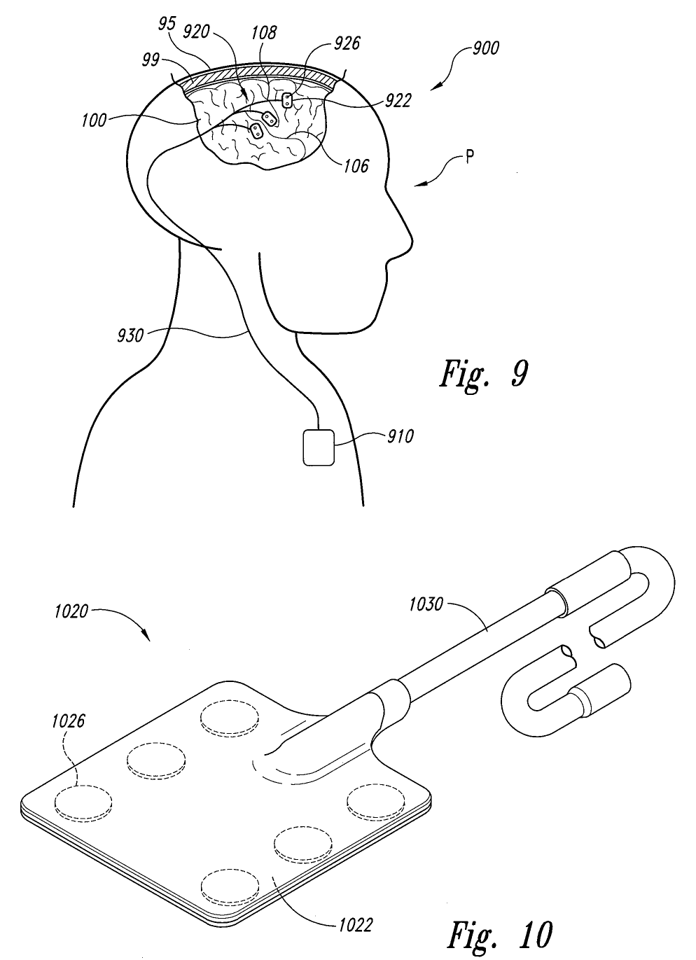

Although shown as E1-E4, additional or fewer stimulation devices may be employed depending upon the nature and/or extent of a patient's neurologic dysfunction, patient condition, neuroanatomical considerations, and/or embodiment details. The stimulation devices E1-E4 may comprise one or more types of signal transfer structures, for example, electrode structures, electrode assemblies, and/or electrical contacts described in various embodiments herein. One or more of E1-E4 may comprise a screw-like or peg-like electrode structure (such as illustrated in FIGS. 4A-6A); a paddle-like electrode structure and/or an electrical contact, for example, as described below in relation to FIGS. 9 and/or 10; and/or another type of structure. Furthermore, various combinations of stimulation device configurations may be chosen to facilitate spatial placements that may enhance a likelihood of affecting particular types of neural structures and/or neural processes in an intended or desired manner.

In view of the foregoing, a neural stimulation configuration in which one or more stimulation devices are positioned or implanted across, between, along, adjacent, and/or relative to portions of a fissure, recess, or groove may facilitate the application or delivery of stimulation signals to one or more portions of a neural population that reside proximate to, upon, and/or within the fissure, recess, or groove. Similarly, a neural stimulation configuration in which one or more stimulation devices are positioned or implanted across, along, and/or adjacent to portions of a vascular structure may facilitate the application or delivery of stimulation signals to one or more portions of a neural population that reside proximate to, beneath, or partially beneath a portion of the vascular structure. Such configurations may enhance neural stimulation efficacy and/or a likelihood of achieving an intended effect when applying stimulation signals having particular stimulation signal parameter characteristics at one or more times, for example, bipolar or unipolar stimulation signals.

Various embodiments of the invention may comprise other and/or additional types of electrical stimulation systems and/or devices configured to facilitate the cross-structure application or delivery of stimulation signals. For example, FIG. 8A is a schematic illustration of a neural stimulation system 800 according to another embodiment of the invention, and FIG. 8B is a corresponding cross sectional schematic illustration of an electrode assembly 820 according to an embodiment of the invention. In one embodiment, the system 800 comprises a pulse generator 850 coupled by a lead wire or link 830 to an energy transfer device or mechanism (E™) 860. The system 800 may further comprise a set of intracranial electrode assemblies 820 implanted relative to one or more neuroanatomical structures 802 under consideration. A neuroanatomical structure 802 under consideration may comprise, for example, a cortical fissure, groove, or recess, and/or a vascular structure, in a manner identical or analogous to that described above. An intracranial electrode assembly 820 may comprise one or more conductive elements, for example, a conductive core 826, carried by an electrically insulating support member such as a head 822 and/or a shaft 824.

In this embodiment, the ETM 860 is configured to apply stimulation signals received from the pulse generator 850 to the patient's scalp 95, for example, in a manner indicated in FIGS. 8A and/or 8B. In some embodiments, the ETM 860 may comprise a conventional adhesive patch electrode. The intracranial electrode assembly 820 may receive stimulation signals through the scalp 95 and convey, deliver, and/or apply such signals to a stimulation site. Particular neural stimulation systems and/or intracranial electrode designs that may transfer stimulation signals from the patient's scalp 95 to a stimulation site are further described in U.S. application Ser. No. 10/891,834, previously incorporated herein by reference.

FIG. 9 is a schematic illustration of a neural stimulation system 900 having an articulated electrode assembly 920 implanted in a patient P to facilitate neural stimulation according to another embodiment of the invention. In one embodiment, the neural stimulation system 900 comprises a pulse generator 910 coupled by a lead wire or link 930 to the articulated electrode assembly 920. Depending upon embodiment details, the articulated electrode assembly 920 may comprise a set of stimulation panels or paddles 922 removably or separably coupled to one another, where each paddle 922 may carry one or more electrodes or electrical contacts 926.

The articulated electrode assembly 920 may be configured to facilitate spatially flexible and/or divergent placement of the individual paddles 922 in relationship to one another at one or more stimulation sites. One or more paddles 922 may be selectively implanted or positioned with respect to a set of neuroanatomical structures under consideration, for example, the lateral sulcus 106, the central sulcus 108, and/or cerebral vasculature to facilitate application or delivery of stimulation signals to portions of a target neural population that may reside proximate to and/or within such neuroanatomical structures. Depending upon the nature of a patient's neurologic dysfunction, patient condition, and/or embodiment details, stimulation paddles 922 may be implanted in the same or different cerebral hemispheres. Any given stimulation paddle 922 may be biased to apply or deliver unipolar and/or bipolar stimulation signals at particular times. Further details relating to various articulated electrode assembly embodiments are described in U.S. patent application Ser. No. 10/707,818, entitled “Articulated Neural Electrode Assembly,” filed Jan. 14, 2004, which is incorporated herein by reference in its entirety.

Some embodiments of the invention may employ a grid or array type electrode structure in association with one or more other types of electrode assemblies or stimulation delivery devices. For example, a grid type electrode structure may be implanted to facilitate the application or delivery of stimulation signals to portions of a gyrus, which may correspond to a neuroanatomical structure under consideration. One or more other electrode assemblies, for example, an intracranial electrode assembly 420a or an articulated electrode assembly paddle 922, may be implanted relative to a neuroanatomical structure under consideration to facilitate establishment of a current path between the grid type electrode structure and the electrode assembly 420a or paddle 922 at one or more times. In certain embodiments, a grid or array type electrode structure may apply or deliver stimulation signals to one target neural population and a cross-structure configuration of stimulation devices may apply or deliver stimulation signals to another target neural population in an alternating or simultaneous manner.

FIG. 10 is a top isometric view of an electrode array 1020 according to an embodiment of the invention. In one embodiment, the electrode array 1020 comprises a support member 1022 that carries a set of electrodes or electrical contacts 1026. A lead wire or link 1030 may couple the contacts 1026 to a pulse generator (not shown) to facilitate the application or delivery of stimulation signals to a neural population. Additional electrode array embodiment details are described in U.S. application Ser. No. 10/112,301, filed Mar. 28, 2002, which is herein incorporated in its entirety by reference.

In some embodiments, imaging techniques may be employed to estimate, determine, and/or assess the location, orientation, condition, and/or nature of particular neuroanatomical structures prior to the implantation or placement of stimulation devices. Relative to neurotopographical structures or features, such imaging techniques may involve, for example, Magnetic Resonance Imaging (MRI).

Depending upon embodiment details, vascular structure imaging techniques may involve ultrasound, CT angiography, magnetic resonance angiography (MRA), laser Doppler flowmetry, and/or other techniques. For CT angiograms, a dye serving as a contrast medium is injected into the arteries of the head or brain for neuroimaging. MRA uses three-dimensional gradient-echo MRI to produce high signal-to-noise ratio images, which can cover extensive regions of vascular anatomy and provide detailed images of blood vessels. A signal generated by a laser Doppler system represents a sampled concentration of moving blood cells in a volume of tissue. Due to the movement of blood cells in vessels, light reflected or scattered by the cells undergo a Doppler frequency shift while light from surrounding tissue remains at its original frequency, thereby providing an indirect method of monitoring microcirculation of blood flow and vasculature characteristics. A vascular structure imaging technique may facilitate or provide for spatial estimation or measurement capabilities such as vessel size or dimension and/or vessel separation.

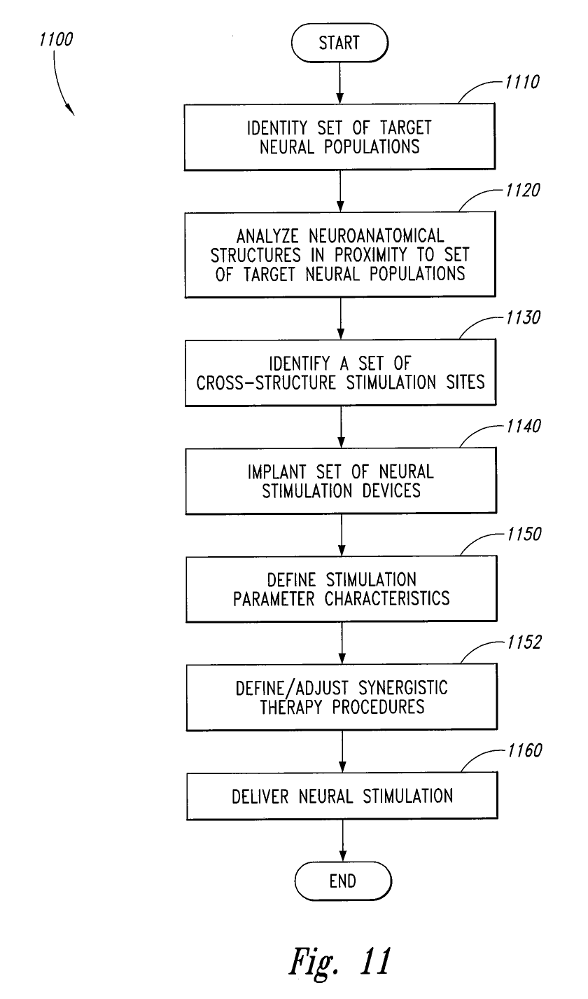

FIG. 11 is a flowchart illustrating an implantation and/or stimulation procedure 1100 according to an embodiment of the invention. In one embodiment, the procedure 1100 comprises a first identification procedure 1110 that involves identifying or determining a set of target neural populations to which neural stimulation may be directed. Depending upon embodiment details, the first identification procedure 1110 may involve a neural imaging procedure (e.g., a procedure involving MRI, functional MRI (fMRI), Diffusion Tensor Imaging (DTI), Positron Emission Tomography (PET), and/or another imaging technique); an electrophysiological measurement procedure (e.g., a procedure involving Electromyography (EMG), Electroencephalography (EEG), and/or Magnetoencephalography (MEG)); an anatomical landmark identification procedure; and/or one or more other procedures. Identification or determination of one or more appropriate target neural populations may depend upon the nature and/or extent of a patient's neurologic dysfunction; patient condition; and/or embodiment details.

The procedure 1100 may further comprise an analysis procedure 1120, which may involve identifying, characterizing, and/or analyzing neuroanatomical structures within, proximate to, and/or at least partially encompassing one or more target neural populations under consideration; and estimating, determining, and/or evaluating one or more target neural population locations, positions, and/or orientations corresponding to such neuroanatomical structures. As indicated above, the neuroanatomical structures may comprise gyri, fissures, grooves, recesses, vasculature, and/or other structures. Depending upon embodiment details, an analysis procedure 1120 may involve a neural imaging procedure.

The procedure 1100 may additionally comprise a second identification procedure 1130 that involves identifying or determining a set of stimulation sites at which corresponding neural stimulation devices may be implanted. The set of stimulation sites may include one or more cross-structure stimulation sites that may facilitate stimulation of portions of particular target neural populations in view of one or more neuroanatomical structures. In certain embodiments, the set of stimulation sites may also include one or more sites at which stimulation devices may be implanted to facilitate stimulation of portions of one or more other neural populations in a manner that is independent or generally independent of particular neuroanatomical structures. Depending upon embodiment details, the second identification procedure 1130 may involve a neural imaging procedure, an electrophysiological measurement procedure, an anatomical landmark identification procedure, and/or one or more other procedures. In certain embodiments, the first and second identification procedures 1110, 1130 may comprise a single procedure.

The procedure 1100 may further comprise an implantation procedure 1140 that involves surgically implanting a set of neural stimulation devices based upon the stimulation site identification procedure 1130. Such neural stimulation devices may comprise one or more electrode assemblies, electrode structures, electrode arrays, pulse generators, lead wires, and/or other devices.

In various embodiments, the procedure 1100 further comprises a first definition procedure 1150 that may involve defining, determining, identifying, and/or establishing a set of neural stimulation parameters that may facilitate the application or delivery of stimulation signals to one or more neural populations under consideration. The first definition procedure 1150 may specify one or more sets of stimulation signal parameters, where each such set may define one or more of a peak amplitude or intensity; a pulse width; a pulse repetition frequency; a polarity; a duty cycle and/or a spatiotemporal activation pattern corresponding to particular neural stimulation devices; and/or other information. In some embodiments, the first definition procedure 1150 may additionally specify one or more stimulation signal application or delivery periods, which may correspond to a particular number of seconds, minutes, hours, days, weeks, months, years, and/or another timeframe.

In some embodiments, the procedure 1100 may also comprise a second definition procedure 1152 that involves defining, determining, identifying, and/or establishing a set of adjunctive and/or synergistic therapy procedures. An adjunctive therapy procedure may involve one or more of a drug therapy procedure; a growth factor and/or neurotrophic agent procedure; a chemical substance procedure; a cell implantation procedure; and/or a behavioral therapy procedure.

Finally, the procedure 1100 may further comprise a therapy application procedure 1160 that involves applying or delivering neural stimulation signals to particular neural stimulation devices at one or more times, for example, in one or more manners indicated above. In certain embodiments, the therapy application procedure 1160 may also involve an adjunctive and/or synergistic therapy, for example, administration of a drug or chemical substance to the patient and/or patient performance of a behavioral therapy during and/or in association with neural stimulation.

From the foregoing, it will be appreciated that specific embodiments of the invention have been described herein for purposes of illustration, but that various modifications may be made without deviating from the spirit and scope of the invention. For example, aspects of the invention described above in the context of particular embodiments may be combined or eliminated in other embodiments. Although advantages associated with certain embodiments of the invention have been described in the context of those embodiments, other embodiments may also exhibit such advantages. Additionally, none of the foregoing embodiments need necessarily exhibit such advantages to fall within the scope of the invention. Accordingly, the invention is not limited except as by the appended claims.

1-60. (canceled)

61. A method for treating a brain disorder, comprising:
implanting at least one electrode device within a patient's skull so that at least one electrical contact of the electrode device is positioned at least proximate to a neuronal cell that is elongated generally along an axis that is generally not perpendicular to the skull proximate to the neuronal cell; and
applying an electrical signal to the at least one electrical contact along electrical field lines that are generally aligned with the axis.

62. The method of claim 61 wherein applying an electrical signal includes applying an electrical signal along electrical field lines that are generally parallel to the skull proximate to the neuronal cell.

63. The method of claim 61 wherein implanting at least one electrode device includes implanting a first electrode device having a first electrical contact and implanting a second electrode device having a second electrical contact spaced apart from the first electrical contact, and wherein applying an electrical signal includes directing an electrical signal from the first electrode to the second electrode, generally parallel to the axis.

64. The method of claim 61 wherein implanting the electrode device includes implanting the electrode device at one from the group of an epidural and a subdural location.

65. The method of claim 61 wherein applying an electrical signal includes applying a unipolar signal.

66. The method of claim 61 wherein applying an electrical signal includes applying a bipolar signal.

67. The method of claim 61 wherein implanting the electrode device includes positioning a first electrical contact adjacent to and on one side of a cranial vascular structure, and positioning a second electrical contact adjacent to and on an opposite side of the cranial vascular structure.

68. The method of claim 61 wherein implanting the electrode device includes positioning the at least one electrical contact proximate to the Sylvian fissure.

69. The method of claim 61 wherein applying an electrical signal includes alternating between a bipolar and a unipolar electrical signal.

70. The method of claim 61, further comprising varying at least one parameter according to which the electrical signal is applied while the electrode device is implanted beneath the patient's skull.

71. The method of claim 61 wherein applying an electrical signal includes transferring the electrical signal from a first conductive structure located external to the patient's scalp, via the patient's scalp to a second conductive structure located beneath the patient's scalp.

72. The method of claim 61 wherein implanting an electrode assembly includes implanting multiple electrode assemblies and wherein the method further comprises coupling the multiple electrode assemblies to a single pulse generator.

73-81. (canceled)
